(19) United States
(12) Patent Application Publication
(10) Pub. No.: US 2013/0340152 A1
(43) Pub. Date: Dec. 26, 2013

(54) AIR FLOW EYE PROTECTION DEVICE AND SYSTEM

(71) Applicant: NICHOLAS CUNNINGHAM, Winter Haven, FL (US)

(72) Inventor: NICHOLAS CUNNINGHAM, Winter Haven, FL (US)

(21) Appl. No.: 13/532,250

(22) Filed: Jun. 25, 2012

(51) Int. Cl. A61F 9/02

(52) U.S. Cl. 2/431

(57) ABSTRACT

An air flow eye protection device includes eye protection goggles having a frame with a plurality of air jets for expelling air in a first direction, temples for securing the frame to a users head, a connector for receiving air from an external device, and an air channel for transporting the received air to the air jets.

An air flow eye protection system includes the eye protection goggles that are in communication with an air supply hose and an air supply device.

### TECHNICAL FIELD

The present invention relates generally to safety equipment, and more particularly to an eye protection device and system capable of utilizing positive air flow to prevent foreign objects from reaching the eyes of a user.

### BACKGROUND

The statements in this section merely provide background information related to the present disclosure and may not constitute prior art.

Blue collar workers such as painters, maintenance personnel, exterminators and construction workers, among others, regularly work in tough environmental conditions. On any given day, these workers are surrounded by hazardous materials such as paint, paint thinner, liquid chemicals, drywall dust, concrete dust, sawdust and other foreign objects that have the potential to become airborne and cause serious injuries to their eyes if contact is made.

There are a number of known protective eyewear devices, such as safety goggles for example, that include a frame for positioning a protective lens in front of the users eyes in order to act as a physical barrier against impact with a foreign object. However, there is a drawback to using conventional goggles when working with airborne materials such as paint spray, and dust, for example. This is because while working, the lens of the goggles often retains the airborne material which continues to build up until the users vision is severely impacted. At that point, the user must stop working, remove the goggles, and attempt to clean them before resuming work. This process often repeats itself several times until the project is complete, or the user stops wearing the goggles out of frustration, thus risking injury to their eyes.

Accordingly, the need exists for an eye protection device and system capable of protecting the eyes of a user without suffering from the drawbacks discussed above.

### SUMMARY OF THE INVENTION

The present invention is directed to an air flow eye protection device and system. One embodiment of the present invention can include eye protection goggles having a frame with a plurality of air jets for expelling air in a first direction, temples for securing the frame to a users head, a connector for receiving air from an external device, and an air channel for transporting the received air to the air jets.

Another embodiment of the present invention is directed to a system that includes the eye protection goggles, an air supply hose and an air supply device.

Yet another embodiment of the present invention can include an air flow eye protection device, that is in communication with an air supply hose and a wearable air supply device.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a frontal view of an air flow eye protection device that is useful for understanding the inventive concepts disclosed herein.

FIG. 2 is a perspective view of the air flow eye protection device in accordance with one embodiment of the invention.

FIG. 3 is a perspective view of the air flow eye protection device in accordance with another embodiment of the invention.

FIG. 4 is a perspective view of an air flow eye protection system in accordance with one embodiment of the invention.

FIG. 5 is a perspective view of the air flow eye protection system in operation, in accordance with another embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

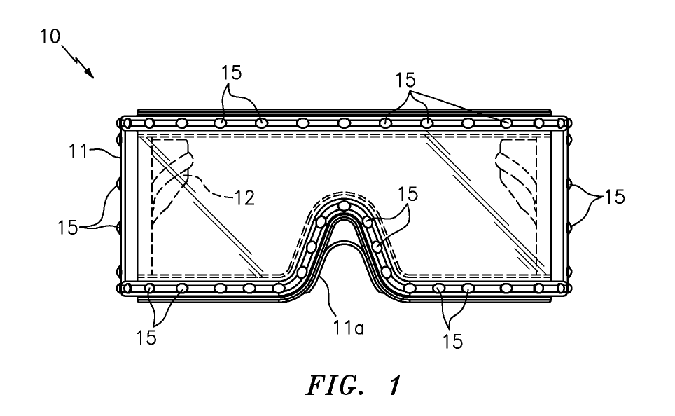

For purposes of this description, the terms “upper,” “bottom,” “right,” “left,” “front,” “vertical,” “horizontal,” and derivatives thereof shall relate to the invention as oriented in FIG. 1.

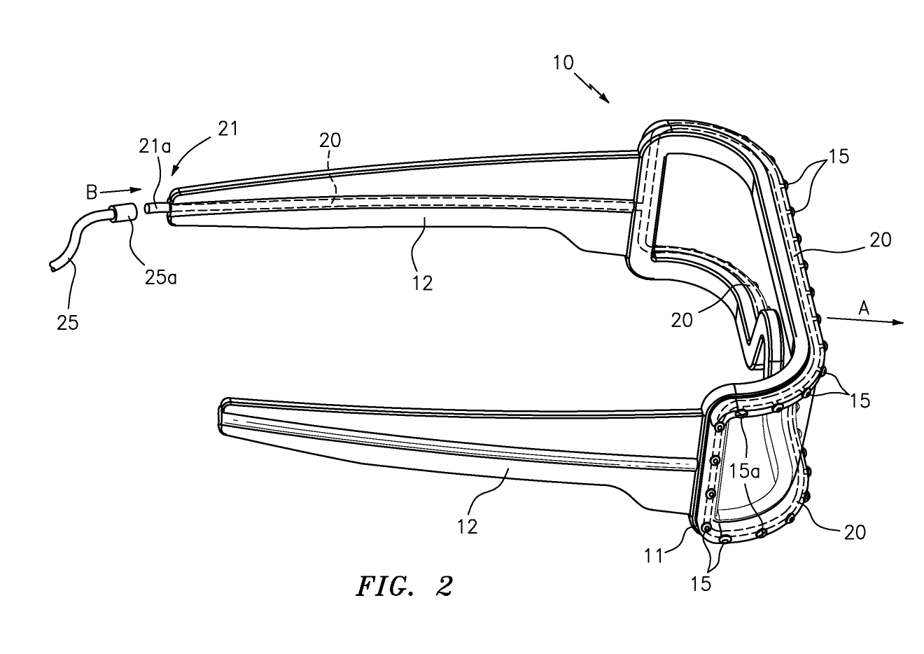

FIGS. 1 and 2 illustrate one embodiment of an air flow eye protection device 10 that is useful for understanding the inventive concepts disclosed herein. As shown, the device 10 can include goggles having a frame 11, a plurality of air jets 15, a frame channel 20 and a connector 21 configured to be secured to an external air hose 25.

The frame 11 can act to position the air jets 15 about the eyes of a wearer. To this end, the frame 11 can include a shape and size similar to conventional goggles and/or glasses for conforming to the face of a user. As shown, the frame can include a curved section acting as a nose stop 11a, and a pair of temples 12 can be conventionally secured to the frame 11 via a hinge, a bonding agent such as glue or resin, or formed as a part of the frame itself through known manufacturing processes. The nose stop and temples working in a conventional manner to allow the user to wear the frame 11 about their head in a manner similar to conventional glasses and goggles.

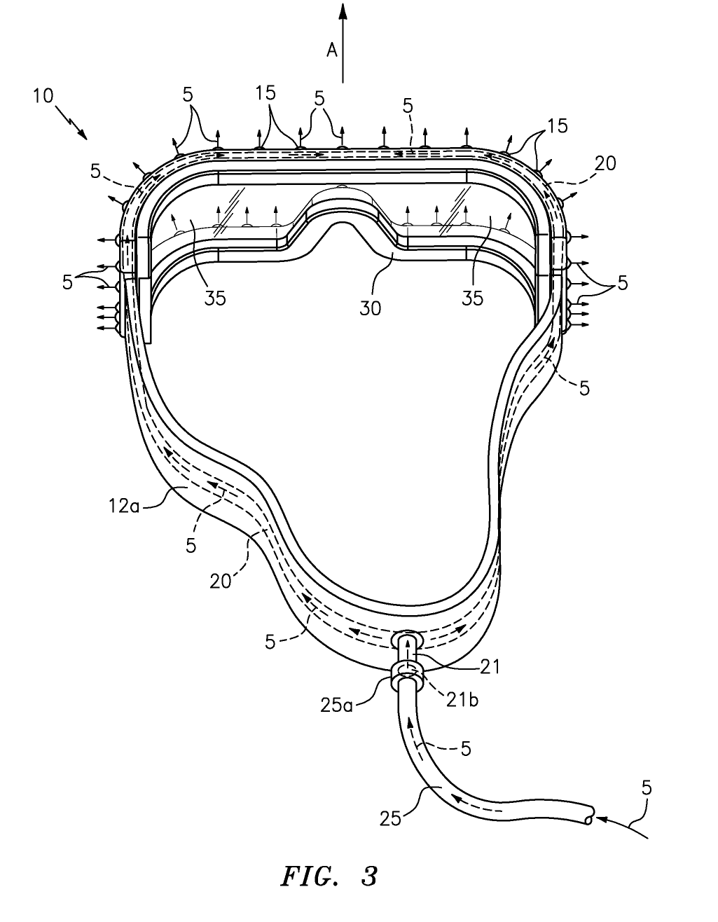

In one preferred embodiment, the frame 11 and temples 14 can be constructed from a robust lightweight material such as plastic, PVC, polycarbonate or other such materials having excellent tensile strength and impact resistant qualities. Of course any number of other known materials and construction methodologies can also be utilized. Moreover, although illustrated above as including a pair of temples, one of skill in the art will recognize that other conventional types of devices such as the adjustable strap 12a (see FIG. 3) can also be utilized without deviating from the scope and spirit of the inventive concepts disclosed herein.

As best shown in FIG. 2, the eye protection device 10 can include a series of hollow tubular elements forming an air channel 20 that is in communication with each of the plurality of valves 15 and a connector 21. The air channel can act to transport compressed air through the device until being expelled by the plurality of air jets 15 positioned along the frame. In one preferred embodiment, the air channel 20 can be embedded within the material of the frame itself, so as to be invisible to a user, and can be constructed from thin plastic tubes having a diameter of approximately [INVENTOR SPECIFIED DIMENSION NEEDED]. Of course other locations, sizes and materials such as polyurethane, polyamide, PE and nylon, among many others, can also be utilized. Construction techniques for embedding materials and channels within objects (such as through the use of a mold, for example) are well known in the art.

Each of the plurality of air jets 15 can act to expel the received air in a direction that is away from the face of the user (see arrow A). To this end, each air jet 15 can include an opening/vent positioned on the frame 11 that is in communication with the air channel 20 in order to receive and expel air. The plurality of air jets 15 can include identical sized vents, or can include a plurality of different sized vents in order to create the desired air barrier.

In one alternate embodiment all or some of the air jets 15 can include a one-way valve 15a in addition to, or in place of the air jets 15 described above. Such a feature can act to expel air in a desired manner, without allowing foreign objects such as dirt, dust and the like from entering the device. One suitable example of a one way valve can include a thin strip of rubber or plastic having an opening through which air can travel in only one direction. One way valves are not new, and no further description will be provided.

The connector 21 can join the air channel 20 to the external air hose 25 in order to allow the device to receive air. As such, one end of the connector 21 can be permanently affixed to the channel 20, and the other end can include an opening 21a for receiving air from the air hose 25. The connector 21 can include any number of sizes and shapes capable of being removably mated with a complementary hose connector 25a (see arrow B), such as a conventional quick connect air coupler, for example. However alternate embodiments having the connector 21 permanently affixed to the external hose 25 are also contemplated.

Although illustrated above as being located within the temples, one of skill in the art will recognize that the connector 21 can be located virtually anywhere on the device 10, such as the frame 11 itself, for example, without deviating from the scope of the inventive concepts disclosed herein.

As described herein, the external hose 25 can include any number of lightweight flexible air hoses suitable for transporting pressurized air. In one preferred embodiment, the external hose 25 can include a hybrid polymer ¼ flexible air hose commercially available from Flexzilla®, however any number of other known air hoses can also be utilized herein.

FIG. 3 illustrates an alternate embodiment of the device 10 that further includes a temple 12a comprising an elastic band, a padded spacer 30 and an impact resistant lens 35. As shown, the elastic band 12a can serve the same function as the separate temples 12 described above, in order to house a portion of the channel 20 and connector 21. The padded spacer 30 can be constructed from any number of soft materials such as foam padding, for example, in order to provide additional comfort to the user while wearing the device.

The optional impact resistant lens 35 can be necessary in some instances for the device to qualify for approval by various government agencies, such as OSHA, for example, and will preferably be constructed from lightweight polycarbonate materials. In either case, when secured to the frame 11 in accordance with known standards, the lens 35 can act as a physical shield for protecting the eyes of the user.

In operation, the device 10 can act to receive air 5 (either pressurized or free flowing) from the air hose 25. The received air 5 can travel throughout the channel 20 until being expelled from the device via the air jets 15. To this end, the expelled airflow coming from the front of the device can form a barrier of moving air in order to prevent hazardous materials (i.e., foreign objects, fumes, soot, and the like) from making contact with the eyes of the user.

Furthermore, when the device is in operation the flow of air emanating from the air jets is constant. As a result, the air 5 acts as a moving shield for capturing airborne articles and actively pushing the same away from the user. To this end, the operation of the device does not allow materials to reach the eyes and/or lens 35 which eliminates the need of the user to constantly stop working and remove accumulated materials.

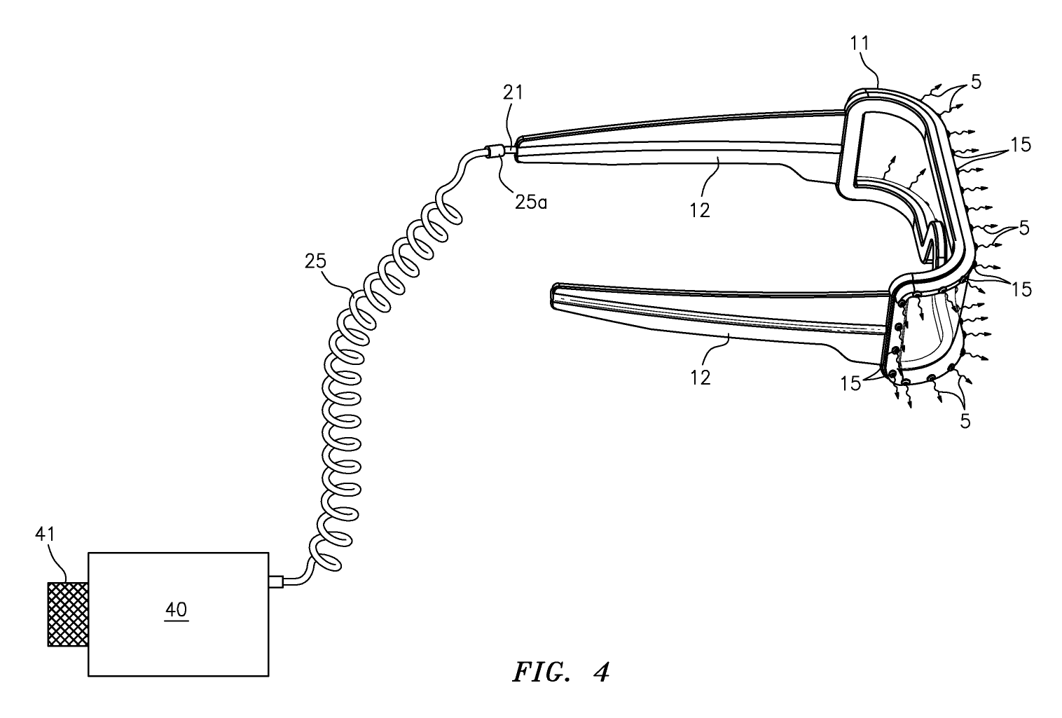

FIG. 4 illustrates one embodiment of an air flow eye protection system 100 that includes an air source 40 that is in communication with the hose 25 and the eye protection device 10, described above.

As shown, air source 40 can preferably include a conventional portable air compressor device capable of generating a user defined volume of compressed fresh air for delivery to the device 10 via the air hose 25. One example of a suitable air compressor includes model C2002-WK commercially available from Porter Cable INC®. To this end, the air compressor 40 can be A/C or D/C powered in order to utilize rechargeable batteries on a construction site. Moreover, an optional air filter/purifier 41 can be secured within the air compressor in order prevent the recirculation of dirty air. Each of these air compressor components are well known in the art and are commercially available. Alternatively, air source 40 can also include any number of other devices such as a compressed air tank, for example.

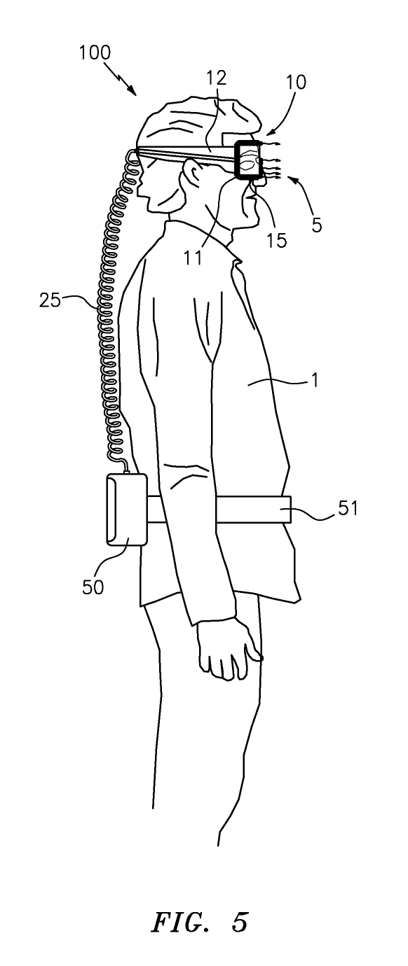

FIG. 5 illustrates an alternate embodiment of the air flow eye protection system 100 that further includes wearable air source 50 having a belt or strap 51 that can be worn by a user 1. Such a feature can allow the system to be utilized within a virtually unlimited range, as the user will not be limited by the length of the air hose 25. As described herein, a wearable air source 50 can include any number of small battery operated air compressors and/or air tanks which can also include optional air purification filters.

Accordingly, the air flow eye protection device and system described above can function to protect the eyes of a user in a novel manner while overcoming the drawbacks of the cited art.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. An air flow eye protection device, comprising:
goggles that include
a frame having a plurality of air jets positioned thereon, each of said air jets being configured to expel air in a first direction,
temples provided at an outer portion of the frame, said temples being configured to secure the frame to a users head,
an air channel that is in communication with each of the plurality of air jets, and
a connector configured to join the air channel to an external device.

2. The device of claim 1, wherein said air channel further comprises:
a plurality of hollow tubular elements configured to transport compressed air.

3. The device of claim 2, wherein each of said hollow tubular members are constructed from at least one of plastic, polyurethane, polyamide, PE and nylon.

4. The device of claim 1, wherein the air channel is embedded within at least one of the frame and the temples.

5. The device of claim 1, wherein the air channel is embedded within each of the frame and the temples.

6. The device of claim 1, wherein each of the plurality of jets comprises a vent.

7. The device of claim 6, wherein each of the plurality of vents include an identical dimension.

8. The device of claim 6 wherein at least one of the plurality of vents includes a dimension that is different from another of the plurality of vents.

9. The device of claim 1, wherein at least one of the air jets comprises a one way valve.

10. The device of claim 1, wherein each of the plurality of air jets comprise a one way valve.

11. The device of claim 1, wherein said connector is configured to mate with a conventional quick connect air coupler.

12. The device of claim 1, wherein said temples comprise
a pair of flexible ear pieces permanently affixed to the frame.

13. The device of claim 1, wherein said temples comprise
at least one of an adjustable band and an elastic band.

14. The device of claim 1, wherein said frame further includes a padded spacer.

15. The device of claim 1, further comprising:
an impact resistant lens secured within the frame.

16. An air flow eye protection system, comprising:
an air source configured to produce a predetermined volume of compressed air;
air flow protection goggles that include
a frame having a plurality of air jets positioned thereon, each of said air jets being configured to expel the compressed air in a first direction,
temples provided at an outer portion of the frame, said temples being configured to secure the frame to a users head,
an air channel that is in communication with each of the plurality of air jets, and
an air hose connector; and
an air hose that is configured to be secured to each of the air source and the air hose connector, said air hose being further configured to transport the compressed air to the air channel.

17. The air flow eye protection system of claim 16, wherein said air source comprises:
at least one of an electric air compressor and an air tank.

18. An air flow eye protection system, comprising:
a wearable air source configured to produce a predetermined volume of compressed air;
air flow protection goggles that include
a frame having a plurality of air jets positioned thereon, each of said air jets being configured to expel the compressed air in a first direction,
temples provided at an outer portion of the frame, said temples being configured to secure the frame to a users head,
an air channel that is in communication with each of the plurality of air jets, and
an air hose connector; and
an air hose that is configured to be secured to each of the air source and the air hose connector, said air hose being further configured to transport the compressed air to the air channel.

19. The air flow eye protection system of claim 18, wherein said wearable air source comprises:
at least one of a battery operated air compressor and an air tank.
